(19) United States
(12) Patent Application Publication
(10) Pub. No.: US 2024/0374209 A1
(43) Pub. Date: Nov. 14, 2024

(54) System and Method for Assessing and Facilitating Improvement of Human Skills

(71) Applicant: Coach AI Limited, London (GB)

(72) Inventors: Matania Zvi KOCHAVI, Tel Aviv-Yafo (IL); Thomas BADER, Darmstadt (DE); Markus SCHLATTMANN, Darmstadt (DE); Michael LEIGSNERING, Darmstadt (DE); Arno MITTELBACH, Darmstadt (DE)

(21) Appl. No.: 18/610,856

(22) Filed: Mar. 20, 2024

Related U.S. Application Data
(63) Continuation-in-part of application No. 18/447,491, filed on Aug. 10, 2023.
(60) Provisional application No. 63/501,483, filed on May 11, 2023.
(60) Provisional application No. 63/501,483, filed on May 11, 2023.

(51) Int. Cl. A61B 5/00; A61B 5/11; G16H 40/67

(52) U.S. Cl. CPC A61B 5/486; A61B 5/1118; A61B 5/1128; A61B 5/4836; A61B 5/744; G16H 40/67

(57) ABSTRACT

The presently disclosed subject matter includes computerized methods and systems for determining performance of human skills of a user by participating in interactive virtual experiences, obtaining user and experience measurements, analysing the performance and determining level of performance of the human skills.

### CROSS-REFERENCE TO RELATED APPLICATIONS

The present application is a continuation-in-part of application Ser. No. 18/447,491, filed Aug. 10, 2023, and also claims the benefit of provisional application No. 63/501,483, filed May 11, 2023, the entire contents of both of which being hereby incorporated herein by reference.

### TECHNICAL FIELD

The presently disclosed subject matter relates to assessing and facilitating the improvement of performance of human skills and, more particularly, to assessing and facilitating the improvement of performance of human skills using virtual activities in computerized systems.

### BACKGROUND

Human skills include various non-technical skills that enable humans to function in society. Human skills may refer to motor, cognitive, social, emotional, language, and adaptive skills. Some examples of skills can include the communication of an individual with others, self-awareness, creative thinking, resilience, understanding non-verbal cues, growth mindset, and other skills. Individuals who encounter challenges in these skills, and desire to enhance their performance in situations that require them, may seek assistance from a therapist, who will collaborate with them to develop and enhance these skills. The therapist might present individuals with situations that could pose challenges, and through continued active engagement in these situations, the individuals will gradually make strides in acquiring the necessary skills. As an example, if an individual is struggling with the skill of resilience, they can be gradually exposed to intimidating situations that may cause them some level of distress. Through this gradual exposure, the individual learns how to respond to these situations, confronts them, and gradually develops the ability to effectively handle the circumstances with calmness.

In practical terms, therapists may provide patients with a treatment plan or a set of recommended exercises to improve their skills. It is expected that patients will diligently carry out these exercises to improve their specific skills. The treatment plan should be implemented during face-to-face sessions with the therapists, as well as by the patients themselves in their free time, in between sessions. By properly executing the treatment plan, patients can experience more rapid and significant improvement in the relevant skills.

However, there are inherent challenges in effectively executing a given treatment plan.

The initial obstacle lies with the patients themselves. Occasionally, patients may struggle to adhere to the treatment plan provided by the therapist due to a lack of motivation. This can occur when the exercises become monotonous and repetitive, and when patients lack continuous guidance and adaptation regarding the execution of exercises and movements. This challenge may be further compounded in the case of young patients, such as children, as their motivation and commitment to executing the treatment plan may be even lower compared to adults.

Furthermore, the therapist responsible for the treatment may only receive partial information from the patients regarding the extent and effectiveness of their execution of the treatment plan. Typically, this information is conveyed by the patients themselves, which introduces inherent subjectivity and lacks objective accuracy. It is provided sporadically and in a non-continuous manner during face-to-face sessions. Another challenge in executing a treatment plan is the absence of objective information and feedback concerning the progress made in performing the exercises. All relevant parties involved, including the patients, therapist, and even the patients' relatives, lack access to this information and feedback. This absence hinders the ability of the parties to update the treatment plan, make necessary corrections, and personalize it to the patient's evolving needs and progress.

Clinics may employ computerized systems aimed at assisting patients in enhancing their human skills. These systems typically offer predetermined stimulation to patients, measure their responses through sensors connected to their bodies, and subsequently relay the measurements to the patient's therapist. However, the requirement for patients to visit the clinic for pre-scheduled appointments with the therapist, along with the need to use specialized sensors available only at the clinic, create difficulties for patients in maintaining consistency in executing any treatment plan due to the aforementioned reasons.

Consequently, it is desired to streamline the execution of treatment plans in a computerized and user-friendly manner. This would allow for efficient feedback regarding the execution of the plan and enable updates to the treatment plan, ultimately enhancing the overall improvement of the skills.

### General Description

The effectiveness of an individual's efforts to improve their human skills through the execution of a treatment plan depends on their perseverance in carrying out the plan, conducting accurate analysis of its execution, and receiving effective feedback. The feedback enables the necessary adaptation to the treatment plan based on the individual's progress.

Current computerized systems require individuals to physically visit the clinic for scheduled appointments with the therapist and utilize specific sensors during exercise sessions. The data obtained from these dedicated sensors is primarily focused on the accuracy of the physical movements performed by the user. In some cases, additional sensors may measure the user's heart rate to track changes in their exertion levels. However, there is a lack of assessment tools that can effectively consolidate different types of data related to various aspects of the individual's performance in the exercises and the treatment plan. Such tools are desired to analyze the data to assess overall performance, and provide comprehensive feedback to either the individual or the therapist.

A computer-implemented method for assessing and determining the level of performance of human skills of a user and facilitating the user to improve these skills, by improving level of performance of these skills, is provided. The method enables a user using a user device, such as a mobile device, to participate in an interactive virtual experience including various activities, for example, a first activity of climbing a mountain, and a second activity of avoiding falling rocks. While the user is participating in the game, a camera, such as the device's camera, can capture the user when participating in the game. Data pertaining to the user can be gathered, such as physical motions of climbing, expressions, and internal functions pertaining to how the user reacted to the rocks that he had to climb, and to the climbing itself. Data on inactivity of the user, such as no physical motion of the user at a certain point in time, can also be gathered as user actions.

Still while the user is participating in the game, data pertaining to execution of the game itself may be extracted, for example how many times the user paused the game, how many rocks the user managed to collect, etc.

The user data and the experience data may be analyzed to obtain analyzed data including analyzed measurements. While the user is participating in the game, the game can be updated, for example, based on the analyzed data, e.g. based on the user's actions, e.g., if the user is outperforming or underperforming, and/or based on the activity measurements.

In some cases, after the execution of a session of experience is completed, combined measurements can be determined. The combination measurements can be based on the user activity in the game, and the experience itself. For example, the user and the experience measurements that were extracted and analyzed. A level of performance of human skills can then be determined based on the analyzed data. The level of performance of human skills can include monitoring and analyzing the skill progression. Alternatively, the skill progression can be determined independently of the level of performance. The level of performance of the user of some human skills and the skill progression can be used to indicate if the user is struggling with certain skills, or has made progress along the course of treatment, e.g., compared to previous performance of the user, and other insights that can be useful in order to assist the user to improve performance of these skills.

Feedback based on the level of performance may be generated and provided to the user, his therapist, or other relevant parties, such as his parents.

The game may be provided to the user as part of a treatment plan formed for the user based on a set of one or more skills that were identified as requiring improvement. Based on the user's level of performance, and as the user makes progress in the game, the treatment plan, the experience (e.g., the game) or activities in the experience, can be updated and adapted to the user's level and progress, resulting in enhancing the overall improvement of the skills of the user.

The analysis of the various types of data, including both the user's actions as well as the execution of the experience, which may reflects the user's overall participation in the experience, is advantageous since the analysis and feedback is monitored in an objective computerized manner, while avoiding the therapist's subjective opinion of the execution, progression can be tracked over time, and the treatment plan can be updated based on the gathered data, to enhance improvement.

The method according to certain embodiments of the presently disclosed subject matter, therefore, motivates real-time experiences, privacy-aware tracking of the treatment plan, and objectively measuring progress and various metrics of the user, to get a detailed status of the improvement of the patient in his human skills, such as physical, cognitive, behavioral, and mental abilities.

The method may further facilitate faster improvement of the patient by using visualization, e.g., by providing a visual stimulus, and interactive experiences including motivating game mechanics which highly motivate patients to participate in such games. This results in greater time spent on these games, and thus in experiences which improve the required human skills. The provided method also changes the traditional repetitive nature of the exercises towards varying visual interactive experiences, which are more immersive and achieve faster improvement of skills.

By monitoring the patient's activity in the game, and his the skill progression, and by adapting the experiences in the game based on the monitored activity, the visual stimulus also provides a more accurate challenge for the patient (physically, mentally, and cognitively).

Another advantage of the presently disclosed subject matter resides in that the game may be executed in a gaming application running on a user's mobile device, where no additional equipment, such as gloves with sensors, or other dedicated equipment, is required. Also, data on the patient performing the game may be gathered, analyzed, stored, and transmitted to relevant parties, such as the therapist, relatives, etc., thus avoiding the need to carry out the required treatment plan in a clinic with a therapist.

According to a first aspect of the presently disclosed subject matter, there is provided computer-implemented method for determining performance of human skills of a user, the method comprising:
providing on a user device an interactive virtual experience in which the user can participate, the experience comprising one or more activities;
while the user participates in the experience, repeatedly executing:
obtaining camera output comprising a video of the user performing one or more user actions;
extracting one or more user measurements from the one or more user actions;
extracting one or more activity measurements from the provided experience;
analyzing at least one of the one or more user measurements and the one or more activity measurements to obtain analyzed measurements; and
adapting at least one of the activities in the experience based on at least the analyzed measurements; and
determining one or more combined measurements based on the analyzed measurements; and
determining level of performance of one or more human skills based on the one or more combined measurements.

In addition to the above features, the computer-implemented method according to this aspect of the presently disclosed subject matter can optionally comprise in some examples one or more of features (i) to (xxvii) below, in any technically possible combination or permutation:
i. the method further comprising:
based on the level of performance, generating feedback; and
providing the feedback;
whereby the provided feedback facilitates the user to improve the level of performance of the human skills by improving performance of at least some of the user measurements.
ii. Wherein at least one activity measurement is not related to success of the user in completing goals of the experience but is used in the determining the level of performance.
iii. Wherein adapting at least one of the activities comprises performing at least one of the following: changing a level of difficulty of at least one of the activities, replacing virtual activities included in the experience, activating and/or deactivating specific activities, adjusting at least one of the activities to assist the user in participating in the experience, providing guidance on experience in the activity, adjusting or triggering audio output, or changing visual appearance of elements included in the experience.
iv. Wherein at least one of the activities involves interaction with a plurality of virtual interactive elements, and wherein adapting at least one of the activities comprises changing the interactive elements in response to a user action.
V. Wherein at least one element includes an avatar configured for mimicking at least one of the user actions.
vi. Wherein the one or more user measurements comprise a body motion.
vii. Wherein the one or more user measurements comprise facial muscles and expressions.
viii Wherein the one or more user measurements comprise internal functions.
ix. Wherein the one or more activity measurements relate to continuous running of the experience.
x. Wherein analyzing the one or more user measurements comprise comparing at least one of the extracted user measurements to a corresponding reference user measurement.
xi. Wherein extracting the one or more activity measurements comprise measuring one or more of performance, goals, or events within the experience, or a combination thereof.
xii. Wherein the analyzing comprises analyzing the one or more user measurements and the one or more activity measurements.
xiii. The method further comprising:
providing the feedback to a third party related to the user.
xiv. Wherein at least one of the user actions comprises a body motion, and wherein the feedback comprises data relating to a more accurate version of the body motion.
xv. Wherein the feedback comprises an avatar configured to illustrate a more accurate version of the body motion.
xvi. Wherein the experience is selected based on a personalized treatment plan generated to facilitate the user to improve one or more human skills by improving performance of at least some of the user measurements, wherein at least some of the activities in the selected experience correspond to the one or more human skills.
xvii. Wherein the treatment plan is automatically generated, based on data pertaining to the user.
xviii. The method further comprising:
providing the feedback to a third party related to the user, wherein the third party is a therapist involved in generating treatment plans;
in response to providing the feedback, receiving a modification input from the therapist; and
adapting at least one of the activities and/or the experience in accordance with the modification input.
xix. Wherein the modification input includes data pertaining to modification in the personalized treatment plan, and prior to the modifying, the method further comprising:
based on the modification input, updating the treatment plan; and
adapting at least one of the activities and/or the experience, based on the updated treatment plan.
xx. Wherein adapting at least one of the activities and/or the experience comprises performing at least one of the following: changing a level of difficulty of at least one of the activities, replacing the virtual activities, activating and/or deactivating specific activities, adjusting at least one of the activities to assist the user in participating the experience, providing guidance on the experience on the activity, adjusting or triggering audio output, or changing visual appearance of elements included in the experience.
xxi. Wherein the user device is a mobile user device comprising a camera.
xxii. Wherein determining the one or more combined measurements comprises processing the analyzed user measurements and the activity measurements in order to assess one or more of the following aspects pertaining to human skills: motion patterns, reaction, performance related to abilities, aggregated measurements over a plurality of experiences, comparison with goals, cognitive and physical abilities, social skills, and behavioral skills.
xxiii. Wherein the analyzing comprises analyzing one or more user measurements using at least one of image analysis and analysis of extracted human body.
xxiv. Wherein determining the level of performance comprises mapping the combined measurements to the one or more human skills.

The method further comprising: determining skill progression for one or more of the human skills.
xxv. wherein determining the skill progression for at least one of the human skills comprises determining the progress in one or more progress markers associated with levels of the at least one skill.
xxvi. wherein the progress levels are selected from a group comprising: completion rate and level success, skill level, effort, counts, metrics or a combination thereof.
xxvii. Wherein a level is considered as reached, after all progress markers associated with the human skill are met.

The presently disclosed subject matter further comprises a computerized system for determining performance of human skills of a user, comprising a processing circuitry that comprises at least one processor and a computer memory, the processing circuitry being configured to execute a method as described above with reference to the first aspect, and may optionally further comprise one or more of the features (i) to (xxviii) listed above, mutatis mutandis, in any technically possible combination or permutation.

The presently disclosed subject matter further comprises a non-transitory computer readable storage medium tangibly embodying a program of instructions that, when executed by a computer, cause the computer to perform a method as described above with reference the first aspect, and may optionally further comprise one or more of the features (i) to (xxvii) listed above, mutatis mutandis, in any technically possible combination or permutation.

According to a second aspect of the presently disclosed subject matter there is provided in a user device, a computer-implemented method for determining performance of human skills of a user, the method comprising:
providing an interactive virtual experience in which the user can participate, the experience comprising one or more activities;
displaying the experience;
while the user participates in the experience, repeatedly executing:
capturing, by a camera included in the user device, camera output of the user performing one or more user actions;
extracting one or more user measurements from the one or more user actions;
extracting one or more activity measurements from the provided experience;
analyzing the one or more user measurements and the one or more activity measurements obtain analyzed measurements;
adapting at least one of the activities in the experience based on at least the analyzed measurements;
determining one or more combined measurements based on the analyzed measurements; and
determining level of performance of one or more human skills based on the one or more combined measurements.

In addition to the above features, the system according to this aspect of the presently disclosed subject matter can comprise the following feature:
i. wherein providing data comprises:
based on one or more parameters relating to human skills, generating feedback; and
providing the feedback;
whereby the provided feedback facilitates the user to improve the level of performance of the human skills by improving performance of parameters indicative of human skills.

### BRIEF DESCRIPTION OF THE DRAWINGS

In order to understand the invention and to see how it can be carried out in practice, embodiments will be described, by way of non-limiting examples, with reference to the accompanying drawings, in which:

FIG. 1 illustrates a high-level diagram of an assessment environment, in accordance with certain embodiments of the presently disclosed subject matter;

FIG. 2 illustrates a functional block diagram of the assessment system, in accordance with certain embodiments of the presently disclosed subject matter;

FIG. 3 illustrates a general flowchart of operations executed in the assessment system, in accordance with certain embodiments of the presently disclosed subject matter;

FIG. 4 illustrates a generalized flow of data between entities and components in assessment environment 100, in accordance with certain embodiments of the presently disclosed subject matter; and

FIGS. 5 and 6 illustrate an exemplary skill 235a, in accordance with certain embodiments of the presently disclosed subject matter.

### DETAILED DESCRIPTION

In the following detailed description, numerous specific details are set forth in order to provide a thorough understanding of the invention. However, it will be understood by those skilled in the art that the presently disclosed subject matter may be practiced without these specific details. In other instances, well-known methods, procedures, components, and circuits have not been described in detail, so as not to obscure the presently disclosed subject matter.

Unless specifically stated otherwise, as apparent from the following discussions, it is appreciated that throughout the specification discussions utilizing terms such as “assessing”, “performing”, “providing”, “obtaining”, “performing”, “extracting”, “adapting”, “analyzing”, “calculating”, “outputting”, “generating”, “receiving”, “updating”, “determining”, or the like, refer to the action(s) and/or process(es) of a computer that manipulate and/or transform data into other data, said data represented as physical, such as electronic, quantities and/or said data representing the physical objects. The terms computer/computer device/computerized system, or the like, should be expansively construed to include any kind of hardware-based electronic device with a processing circuitry (e.g., digital signal processor (DSP), a GPU, a TPU, a field programmable gate array (FPGA), an application specific integrated circuit (ASIC), microcontroller, microprocessor etc.), including, by way of non-limiting example, computerized systems or devices such as disease data storage and retrieval system 100 disclosed in the present application. The processing circuitry can comprise, for example, one or more processors operatively connected to computer memory, loaded with executable instructions for executing operations as further described below.

The terms “non-transitory memory” and “non-transitory storage medium” used herein should be expansively construed to cover any volatile or non-volatile computer memory suitable to the presently disclosed subject matter.

The operations in accordance with the teachings herein may be performed by a computer specially constructed for the desired purposes, or by a general-purpose computer specially configured for the desired purpose by a computer program stored in a non-transitory computer-readable storage medium.

Usage of conditional language, such as “may”, “might”, or variants thereof, should be construed as conveying that one or more examples of the subject matter may include, while one or more other examples of the subject matter may not necessarily include, certain methods, procedures, components, and features. Thus, such conditional language is not generally intended to imply that a particular described method, procedure, component or circuit is necessarily included in all examples of the subject matter. Moreover, the usage of non-conditional language does not necessarily imply that a particular described method, procedure, component, or circuit, is necessarily included in all examples of the subject matter. Also, reference in the specification to “one case”, “some cases”, “other cases”, or variants thereof, means that a particular feature, structure, or characteristic described in connection with the embodiment(s), is included in at least one embodiment of the presently disclosed subject matter. Thus, the appearance of the phrase “one case”, “some cases”, “other cases” or variants thereof does not necessarily refer to the same embodiment(s).

It is appreciated that certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

Human skills may refer to skills pertaining to motor, cognitive, social, emotional, language, and adaptive skills. Some examples of human skills may include communication, self awareness, creative-thinking, resilience, understanding nonverbal cues, growth mindset, gross motor, fine motor, visual perception and processing, motor planning and control, executive functioning, and other skills. Consider an example of an individual struggling with the skill of resilience. The individual may struggle to recover quickly and effectively after experiencing failures. Enhancing the resilience skill may involve the ability of the individual to effectively manage stress and pressure. An example of an experience to assist the individual in enhancing his resilience skill may involve a game featuring a virtual environment inspired by nature or the wilderness, where the lower level of difficulties of the game features a calm nature, but, as the user makes progress, the nature may be featured with a more intimidating elements, gradually forcing the user to confront them. Monitoring measurements pertaining to the user and the game during the user's participation may assist in obtaining data on the level of performance of the user of the skill of resilience, if the user makes progress.

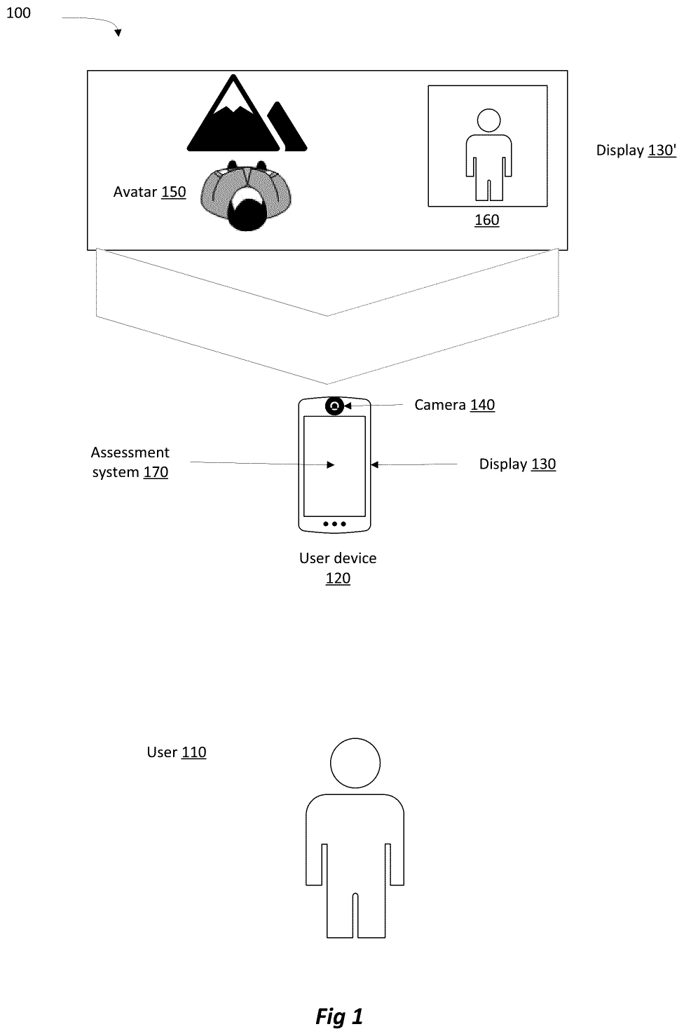

In accordance with certain embodiments of the presently disclosed subject matter, there is provided a method for determining performance of human skills of a user. Bearing this in mind, attention is drawn to FIG. 1 illustrating a high-level diagram of an assessment environment 100, in accordance with certain embodiments of the presently disclosed subject matter. Assessment environment 100 is configured to enable the method for determining the level of performance of human skills of a user 110. In some examples, environment 100 may include several entities, some operatively communicating via a network. A user 110, also referred to throughout the application as a “patient”, may operate a user device 120 to participate in an interactive virtual experience. The experience may comprise one activity, or may include a plurality of activities. In some examples, an experience may be referred to as a game including one or more activities. For example, a game may include a virtual environment of a desert with a mountain, where one activity of the game can include a task for the user to climb the mountain. Another activity in the game can be the task for the user to collect or avoid rocks that fall from the mountain during his climbing. The user participating in the game should climb the mountain, by performing physical motions typical for climbing a mountain, such as raising the hands and pushing himself up, and/or by moving the legs accordingly. The user may also perform physical motions typical for avoiding hitting the rocks or, alternatively, selecting them, per the task of the game.

The experience may be provided to the user device 120 and user device may provide the experience to the user, including displaying the experience on the display. In some examples, the experience that is presented to the user can include high end graphics and game mechanics that are typically used in gaming. An activity in the experience may be generated in a manner that addresses one or more parameters and variations that emphasize different aspects of human skills. For example, one variant or parametrization may focus more on the physical aspect, e.g. the user's motion, while another one focuses on the cognitive aspect. For example, the activity of avoiding falling rocks was generated to address user's physical abilities to move the body to avoid the rocks, as well as the cognitive aspect of ability to recognize the danger of a falling rock and the need to avoid it.

Another example of a typical calming exercise in psychotherapy is brushing gently with the hand along the arm from the shoulder to the fingertips. A gamified version can be created where the avatar has some golden dust on the arm that needs to be gently brushed away. The motion analytics can be used to understand if the exercise is done correctly. Also, even before starting the exercise, emotion and behavior analytics can be used in order to understand if a calming exercise should be suggested to the user.

Also, each experience or game may include multiple levels that constitute a difficulty progression for a relevant skill. The multiple levels can be used when adapting the experience while the user participates the game, or later in a following session that is provided to the user, e.g. based on the feedback that was generated for a certain session.

Different experiences can be wrapped together for a longer sequence, e.g., to create a higher level of motivation and combine different aspects together, for example, individual interactive experiences can be wrapped in a story. Also, those skilled in the art will also readily appreciate that the recitation of a game should not be considered as limiting, and an experience can be any flow of events e.g., in an entertaining activity, without necessary posing limiting rules or guidance typically appearing in games, on the user, where an experience may be wrapped in e.g., a story, as mentioned above.

The user device 120 illustrated in FIG. 1, which may be a smartphone, may include a display 130 such as the screen of the user device, or any display that is visible to the user. The content on the display 130 may be reacting in real-time (e.g., using CPU, GPU, and/or NPU of the user device 120). The user device 120 may also include a camera 140 which may be configured to capture the user 110 participating in the game, e.g., performing the motions. Display 130′ included in FIG. 1 illustrates exemplary content displayed on the display 130, e.g., the desert with the mountain. The game may include one or more virtual interactive elements, while some activities in the game involve interaction with these elements. The elements are configured to change in response to a user action when participating in the activity, or respond to the user's actions. A virtual interactive element of an avatar 150, either human or non-human, may be included in the virtual environment of the game and may be displayed on the display 130. The avatar 150 may change by mimicking at least one of the user actions, including the user's physical motions, emotional expressions, or the user's breathing patterns, and emotional expressions as estimated from the facial muscles and appearance, all as captured by the camera of the user device 120. For example, if the user 110 is performing climbing motions, the display 130 may react in real-time and will display the avatar 150 climbing the virtual mount. The display 130 may also display any virtual interactive elements such as falling rocks from the mountain.

The display 130 may also include an area 160 in which the user 110 is shown, as captured by camera 140, optionally, in real time.

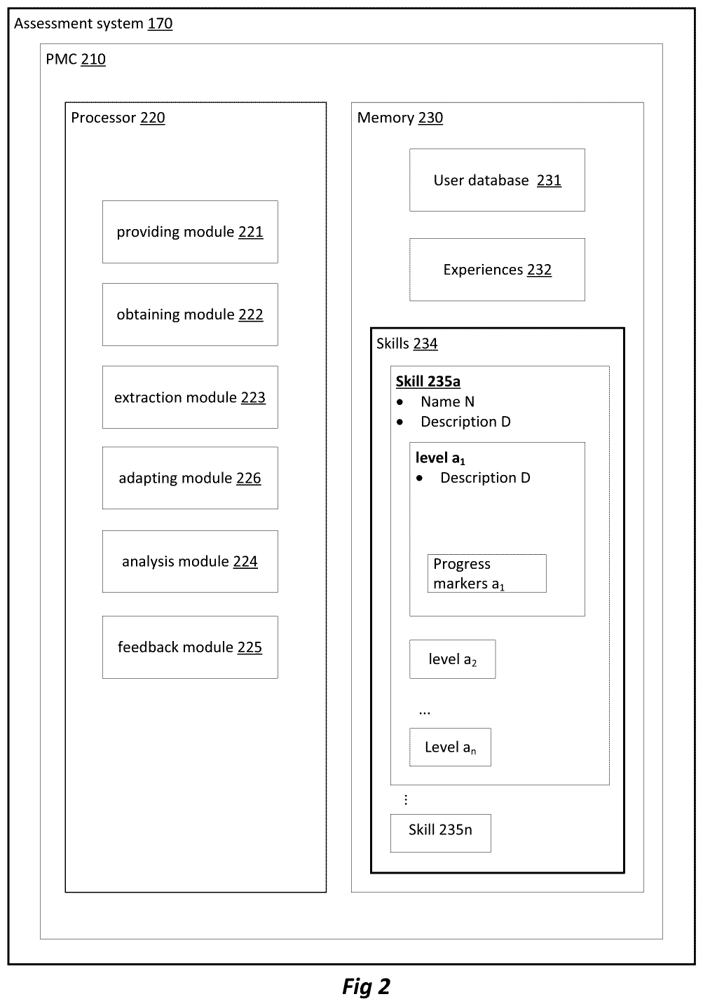

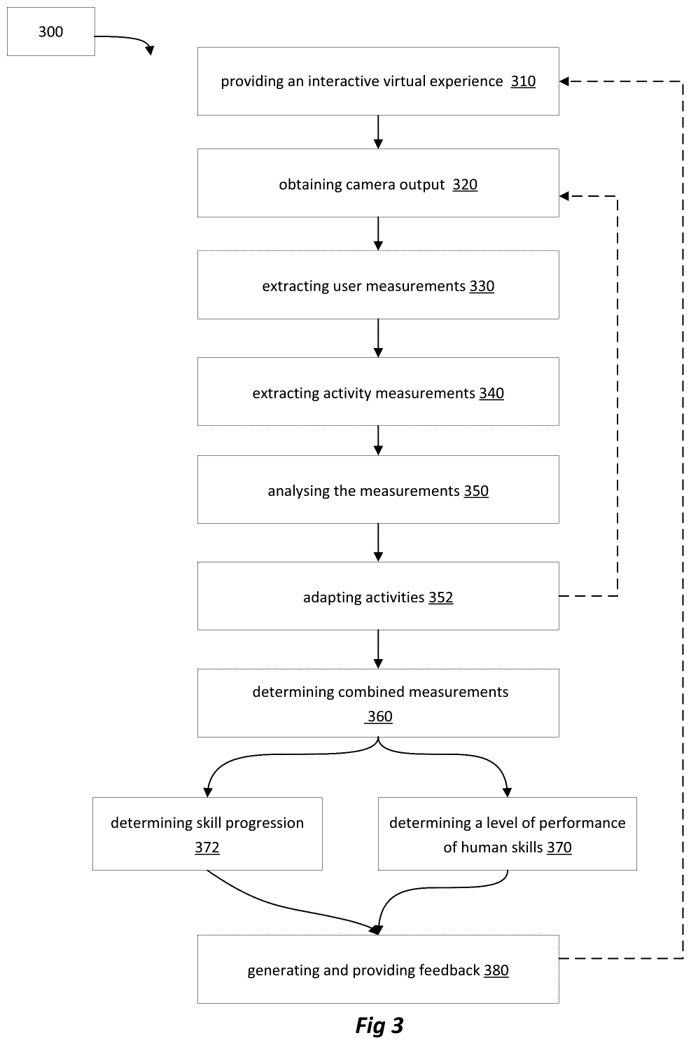

An assessment system 170 included in FIG. 1 is configured to provide the experience to be displayed on the user device 120. While the user participates in the experience, the camera 140 can capture a video of the user 110. The assessment system 170 is configured to obtain the video, extract relevant data and measurements from the video, and analyze them to calculate a level of performance of the user 110, and, optionally, provide feedback based on the level of performance of the user 110. In some examples, the assessment system 170 can provide feedback to the user 110 or to another party related to the user 110 including insights on how the user 110 participated in the experience. Further details pertaining to the assessment appear below with respect to FIGS. 2 and 3.

Although assessment system 170 is included in user device 120, it should not be considered as limiting, and assessment system 170 can be operationally connected to the user device 120 and may also be located outside or partially outside the user's device 120, and is configured to communicate with the user's device 120 to send and receive data. For example, assessment system 170 can send data indicative of the experience to the user device 120 to be displayed on the display 130, and is configured to receive data on the video captured by camera 140 and optionally other data pertaining to the experience.

Reference is now made to FIG. 2 illustrating a functional block diagram of the assessment system 170, in accordance with certain embodiments of the presently disclosed subject matter. The illustrated assessment system 170 includes a processor and memory circuitry (PMC) 210 comprising a processor 220 and a memory 230. The processor 220 is configured to execute several functional modules in accordance with computer-readable instructions implemented on a non-transitory computer-readable storage medium. Such functional modules are referred to hereinafter as comprised in the processor 220. The processor 220 can comprise a providing module 221, an obtaining module 222, an adapting module 226, an extraction module 223, an analysis module 224, and a feedback module 225.

The memory 230 can store data pertaining to the users in user database 231, such personal data including age, gender, family data, the skills each user desires to enhance, treatment plans associated with each user, history of progress, and other data. The memory 230 can also store experiences 232 which may store data pertaining to various games and the correspondence between the activities in each game to human skills, so that a game may be selected to a user, based on a set of one or more skills that he is required to enhance.

The memory 230 can also store Skills 234, including one or more non-technical human skills 235a-235n. Each of the skills may refer to human skills that enable humans to function in in every day life. A skill 235a of human skills 235a-235n may refer to motor, cognitive, social, emotional, language, and adaptive skill as described above. The skill 235a may consist of a name and a description, and a predefined number of levels a₁-aₙ. The skill level a₁ may have a description. A certain level may be determined as a target level for a particular user 110, e.g. as a part of the treatment plan, such that achieving that target level is considered as aₙ achieved goal in the treatment plan. Each of the skill levels a₁-aₙ may also be associated with a respective set of one or more progress markers a₁. The progress markers may indicate measures to evaluate the progress of a patient in a skill level. A single progress marker may be used to determine when a skill level is reached, so the user 110 may move to the next level. Different types of progress markers exist. Progress markers can be defined in tabular form. A level can be reached once all progress markers are met, either all at once or over time.

Some examples of progress markers types include:
completion rate, level success: progress markers can be defined as conditions based on completion rate of certain set of games/levels, success (as opposed to failure) in individual levels or a set of levels. These parameters may be measured as part of the activity measurements.
skill level: progress markers can contain conditions that refer to achievement of a certain level in a different skill. For example, achievement of level 2 in skill A can be one of the progress markers for achieving level 3 in skill B.
Effort: overall playtime or playtime in certain games. A progress marker for example can be defined based on how much time the user played a certain game or certain set of games. Playtime is measured as part of activity measurements.
Counts: count of events that happened in games such as certain obstacles hit, errors made, items collected. The information may be collected as part of activity measurements and can be used to define progress markers. For example, in order to achieve a certain progress marker it might be defined to as requirement to hit less than 20% of all obstacles presented in a set of games or collect at least 70% of all collectable items presented in the games.
metrics: progress markers can be defined based on conditions including performance metrics. These can be based on user measurements, analyzed measurements or combined measurements as defined below.
a combination of any of the above.

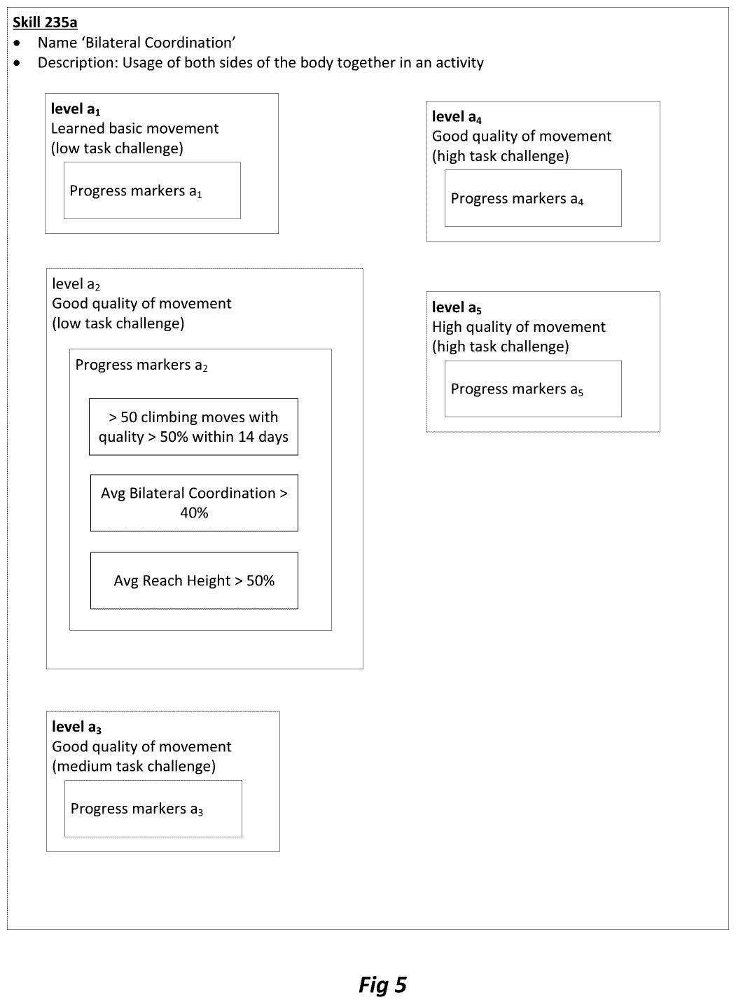

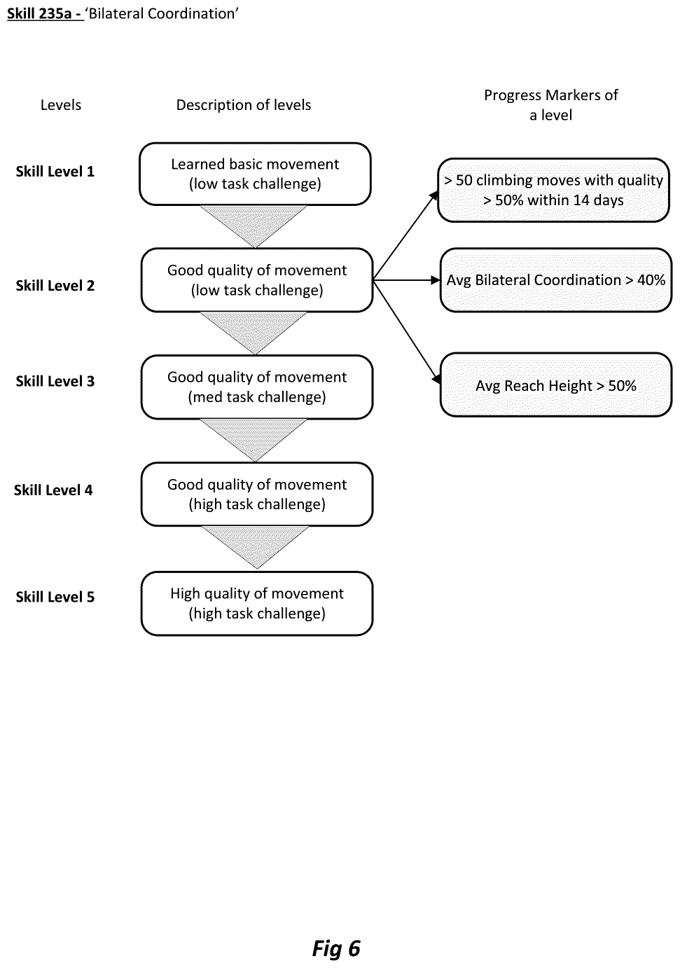

Reference is made to FIGS. 5 and 6 illustrating an exemplary skill 235a named ‘Bilateral Coordination’, stored in memory 230, in accordance with certain embodiments of the presently disclosed subject matter. Bilateral Coordination skill 235a may measure the usage of both sides of the body together in an activity. Bilateral Coordination skill may be defined with 5 skill levels a₁-a₅, where each level may be associated with progress markers. For example, as illustrated in FIGS. 5 and 6, level a₂ of ‘good quality of movement (low task challenge)’, may be associated with a set a₂ of 3 progress markers:
1. >50 climbing moves with quality>50% within 14 days;
2. Avg Bilateral Coordination>40%; and
3. Avg Reach Height>50%.
   The user's participation in the experience is processed and evaluated. If it is determined that the user 110 performed in the experience more than 50 climbing moves having quality of above 50% within 14 days, and the user's average Bilateral Coordination>40% (determined by a performance metric based on user measurements, reflecting how coordinated both sides of the body move), and that the user's average reach height>50% (measured based on user measurements determining how high arms reached during required movement to operate the game), then it is determined that all progress markers are achieved and that the level a2 is achieved. In the next participation of the user 110 in a next experience, the next level of experience may be provided to the user, e.g., level 3. The user's participation in the next experience will be evaluated vis-a-via the respective level 3's progress markers (not illustrated in FIGS. 5 and 6). Processing and evaluating the user's participation is described further below with respect to FIG. 3. Alternatively, the user 110 can stay for more than one experience on the same level, until all progress markers of respective levels are achieved.

Referring back to FIG. 2, in some examples, processor 220, e.g., using providing module 221, is configured to provide on the user device 120 an interactive virtual experience, such as a game. For example, providing module 221 is configured to provide to the user device 120 an experience to be displayed on the display of the user device 120. The user 110 can participate in the experience including the one or more activities.

Camera 140 can capture a video of the user 110. While the user 110 is participating in the game, several components in processor 220 may be repeatedly configured to execute several actions. The obtaining module 222 is configured to obtain from the camera 140 a camera output comprising the video of the user 110 performing one or more actions. A user action can be any action of user 110, including performing physical motions, such as raising his hands. However, a user action can also include non-performing movements, such as if the user stands still and does not move. User actions can also relate to movements of facial muscles of the user 110 when having expressions.

Also performed repeatedly, while the user 110 is participating in the experience, the extraction module 223 is configured to extract data pertaining to participation of the user 110. The extraction module 223 is configured to extract user measurements from the action of the user 110 as captured in the video. User measurements can include body motion, facial muscles and expressions, and internal functions.

The extraction module 223 is further configured to extract one or more activity measurements from the experience that is running, while the user is participating in it. Such activity measurements can include data pertaining to the experience itself, e.g., measuring one or more of performance, goals, or events within the executed game, or a combination thereof, such as if the user paused the game many times, how many elements interacted with the user, the difficulty level of the experience, etc.

In some cases, still while the user participates in the game, the analysis module 224 is configured to analyze the user measurements or the activity measurements, or a combination thereof, to obtain analyzed measurements. In some examples, the analyzed measurements can include for example, analysis of the motions of the user (user measurements), e.g. how well he performed in the climbing motions. The analyzed measurements can also include activity measurements such as how many rocks did the user managed to avoid. Yet, in some examples, the analyzed measurements can include a combination of the analysis of the user measurements and the activity measurements, e.g. how the user performed in the activity. As an example, a pause in the user motion together with falling rocks at certain timing in the experience may indicate on stress of the user. Further details of the analysis appear below with respect to FIG. 3.

The analyzed measurements, for example, either or both of the user measurements and the activity measurements can then be used to adapt at least one of the activities in the experience. For example, based on a combination of the user and activity measurements that indicates stress of the user, a virtual background of an intense activity can be replaced with a calmer background. Also, the experience can be adapted as a result of the user 110 controlling the experience. The user's actions can affect an avatar 150 included in the experience, such that the avatar 150 mimics the user's actions. Adapting one or more of the activities may be performed e.g., by adapting module 226. Further details of the user measurements appear below with respect to FIG. 3.

In some examples, once the experience is over, analysis module 224 is configured to determine one or more combined measurements. Determining the combined measurements can be performed in a similar manner to the analysis of the measurements described above. Determining the combined measurements may be based on either of the analyzed user measurements and the one or more analyzed activity measurements, or on a combination thereof.

In addition, the analysis module 224 is configured to determine a level of performance of one or more human skills based on the one or more combined measurements. For example, the user's correct physical motions, together with continuity of the activity, can be analyzed, and the performance level of one or more human skills may be determined based on the analysis. Examples of how the measurements are analyzed appear below with respect to FIG. 3.

The feedback module 225 is configured to generate feedback based on the level of performance. The feedback may later be displayed on the display 130, or may be provided to the user 110 or other parties.

It is noted that the teachings of the presently disclosed subject matter are neither bound by the assessment environment 100 described with reference to FIG. 1, nor by the assessment system 170 described with reference to FIG. 2. Equivalent and/or modified functionality can be consolidated or divided in another manner, and can be implemented in any appropriate combination of software with firmware and/or hardware, and executed on a suitable device. For example, camera 140 can be comprised in user device 120, or may be a separate component operatively connected to the user device 120. Also, display 130 may be a separate component to user device 120, such as a TV screen. Also, in some examples, the assessment system 170 can be part of user device 120, or outside, and communicate with user device 120 to send and receive data.

Those skilled in the art will also readily appreciate that the data repositories/databases in memory 230 can be consolidated or divided in another manner; databases can be shared with other systems or be provided by other systems, including third party equipment.

Reference is now made to FIG. 3, illustrating a general flowchart of operations executed in the assessment system 170, in accordance with certain embodiments of the presently disclosed subject matter. In some examples, the operations can be performed by entities in the assessment environment 100 and assessment system 170 illustrated with reference to FIGS. 1 and 2.

In some cases, a computer-implemented method for determining performance of human skills of a user 110 is provided. In some examples, the process may start by determining one or more experiences suitable for the user 110. The experiences can be derived from a treatment plan determined, e.g. by a therapist, for the user 110. The therapist can determine one or more skills that were identified as requiring improvement for the particular user 110, and may also determine a starting level and a target level for each of the skills representing the starting point of the user 110 and the level which should be achieved by him, respectively. An experience may be generated in accordance with the determined treatment plan or the predefined set of skills determined for the user 110. The predefined skills can be selected from skills 235a-253n stored in skills 234 in memory 230. Optionally, a starting or progressive level selected from levels a₁-aₙ is defined for each of the selected skills 235a-253n. The experience may be generated in accordance with the selected levels for each of the selected skills.

To determine the performance of the human skills in some cases, providing module 221 may provide on a user device 120 an interactive virtual experience (block 310). The experience can be derived from the determined treatment plan, and can be configured to reflect the starting level determined for a user. In some examples, the experience is a computerized game. The experience may comprise one or more activities. For example, the experience can be a computerized game in which the user can climb a mountain constituting the first activity. In another example, the experience can also task the user with collecting rocks on the way up, constituting a second activity in the experience. As described above, an activity in the experience may be generated in a manner that addresses one or more parameters and variations that emphasize different aspects of human skills, for example, the physical aspect or cognitive aspect. Also, the experience or the game may be a single experience or a plurality of experiences, as well as one or more levels of difficulty within the experience, such as individual interactive experiences that are wrapped in a story.

The user 110 operating the user device 120 may participate in the experience, e.g., in the game, and experience physical, cognitive, behavioral, and mental challenges, by participating in activities of the game that were generated in a manner that addresses different aspects of human skills. In some examples, if the user 110 already participated in an experience, and his level of performance may be determined. In addition, the skill progression, achieved levels and the target level may be determined. Any repeated participation of the user 110 in an experience can also be based on the determined skill progression and any target level of progression that is defined for the user 110. Some activities may include various exercises that the user 110 is tasked to perform, such as both climbing the mountain and avoiding the rocks.

The user device 120 may include a video camera 140 that may capture a video of the user 110 participating in the game and performing a plurality of user actions. The captured video, e.g., the camera output, may include a plurality of user actions, including e.g., a plurality of physical motions, facial expressions, and internal functions. While the user participates in the experience, PMC 210 is configured to repeatedly execute several stages of the method. Obtaining module 222 may obtain from the camera 140 camera output of the user (block 320). The camera output comprises a video of the user 110 performing one or more user actions. A user action can be any action of user 110, including performing physical motions, such as raising his hands. However, a user action can also include non-performing movements, such as if the user stands still and does not move. User actions can also relate to movements of facial muscles or the user 110 when having expressions.

Extraction module 223 can analyze the user actions in the camera output, and extract data pertaining to at least one user measurement from the user actions (block 330). For example, extraction module 223 can analyze the human body in one or more images of the video to extract user measurements. Several categories of user measurements can be extracted: body motion, facial muscles and expressions, and internal functions. The category of body motion user measurements can include data pertaining to the pose of the hands, fingers, head, eyes, joints, metrics (individual measures like an angle between certain body joints or dynamic measures like speed of a joint), interactions (the interactions of the user 110 with virtual elements appearing in the game), and other human actions such as a squat, clapping with the hand, thumbs-up gestures, and such. The category of facial muscles and expressions as user measurements can include data pertaining to emotional reactions, e.g., rising the corners of the mouth or constantly moving the eye gaze to the side. The category of internal function user measurements can include data pertaining to bio signals, such as heart rate, heart rate variability, breathing patterns and rate, and others. Extraction module 223 can extract the internal functions using known methods.

Following are some examples of analysis techniques that may be performed by extraction module 223 on the captured video to extract user measurements, with respect to the above categories of data:

### I. Image Analysis

a. The captured video from the camera 140 comprising images may be analyzed using deep learning technology. Details about the human body may be determined, and, optionally, additional visible elements (e.g., movable objects and environment).
b. The human body may be tracked along the images of the video, e.g., using pose estimation techniques. For example, pose estimation can determine the orientation of the individual body joints to estimate a better representation of the human pose. Pose estimation can also determine the individual bone length based on a sequence of pose estimations.
c. In some examples, based on the pose of the user as captured in the video, extraction module 223 may zoom into the image in the video, to determine and extract also details on the face and/or the joints of the hands in order to determine facial muscles and expressions.
d. Based on joint positions in the image and visual cues, a 3D estimation of one or more joints can be performed to enable view-independent analysis, and extract the human body from its environment as captured by the camera.
e. In addition to body joints, additional data may be extracted and analyzed, e.g., facial features.
f. The environment can also be processed, e.g., tracking objects in the environment and interactions with them, for example to extract data on whether the user is kicking a ball. Objects in the environment can be detected and tracked using known methods for object detection and tracking. Through image classification the environment can be classified to, e.g. be., being in a usual home versus being outdoors or at a beach. The rough 3D structure of the environment can be taken into account by applying SLAM (Simultaneous Localization and Mapping) and while using the user device 120 sensors.
g. In addition to kinematic elements of the body, internal functions of the body can be automatically extracted from the video, for example measurement of heart rate and heart rate variability by means of analyzing color variations in the image over time, or breathing patterns by analyzing subtle movement patterns of the body.
   II. Analysis of the extracted human body
On top of the image analysis, certain models can determine motion states, actions, activities, and interactions, such as certain body postures or body moves like a t-pose, a bi-lateral climbing motion, or pinch gestures with different fingers. The data may be analyzed, e.g., using a sequence of images, e.g., in aₙ on-line fashion, to give results in real-time.
This analysis to extract user measurements can be done, e.g., using the following technologies:
   a. a data-driven learning approach based on previously collected data, such as certain poses potentially including certain kinds of a motion. For example, the collected data can include examples of the correct motion and examples of different incorrect motion. This motion might further be tagged qualitatively (e.g., very accurate motion, high stretching motion, certain typical mistakes) or quantitatively (e.g., by providing certain metric values that describe the motion itself, or a certain metric like an angle of motion, maximal reach, or the like). For example, at least one user measurement can be compared to a corresponding reference user measurement.
   b. Template-based similarity/matching to identify certain poses or states within an action. The template can be inserted as an input, e.g., as one or more human poses. A treatment plan or a set of exercises in a game may not necessarily compare correct or in-correct movements, but also gradual and quantitative assessment.
   c. This way, it may be possible to facilitate monitoring the actions of the user, the time that the user performs a certain action, and what type of action (e.g. performs a squat, lifts an arm to a certain height, waves with the hands, performs a finger pinch gesture, etc.)
   d. Interactions with other persons, objects, and the environment, can be determined and classified (e.g., kicking/touching a ball with a certain body part)
   e. In addition to understanding the time and types of actions, motion or activity that has occurred, certain metrics are determined that can be used in further analysis:
      i. A metric can be an individual measure like an angle between certain body joints, a position relative to the floor, or other joint positions
      ii. A metric can also be a dynamic measure like speed of a joint, angular velocity, or the amount of movement
      iii. Metrics can be calculated in a continuous fashion, or based on the time-context of a human action/interaction, as analyzed in the captured video and images of the user.
   f. Complex or application specific actions can be defined such that, e.g.
      i. Combine several metrics and/or actions (e.g., bilateral coordination, reach height, or fluency)
      ii. Are learned using a data-driven approach using previously collected data. Hence, a sequence of poses may be mapped to a certain (learned) metric score.

Extraction module 223 can also extract data pertaining to at least one activity measurement from the provided experience (block 340). For example, extraction module 223 can receive data on execution of the game, and analyze the execution to extract activity measurements pertaining to measuring one or more of performance, goals or events within the executed game, or a combination thereof. For example, the following data can be extracted: one or more activity measurements relating to continuous running of the experience, e.g. how many times the game was paused or how many times a different level was selected by the user while another level is running, data pertaining to the trajectory of game character, such as location, speed, and movement direction of an avatar, or a game character over time, collision or collection of interactive elements, other triggers that affect the game (cut scenes, pauses, etc.), status of the game elements that can affect the user, for example, status of elements that induce stress, distraction, etc. For illustration, consider that the experience includes a scripted or randomly activated game element that triggers stress for the user 110. This could be e.g., time pressure, by stating “your oxygen level is critically low, replenish withing 10s”. Another example in the experience is a “sand storm” which reduces the visibility for the user 110 and makes navigation more challenging. Activity measurements can include parameters pertaining to these elements, such as the time that they were in certain status, duration in each status, etc.

It should be noted that, in some examples, the activity measurements are not related to success of the user 110 in completing goals of the experience, as in regular games, whether the user finished the current level and reached the next. The activity measurements may be used to determine the level of performance of the human skills. For example, assume that the user 110 is tasked with collecting only blue rocks, while rocks of several colors appear in the game. One activity measurement may include the number of red rocks the user collected, irrespective of how many blue rocks he collected. Assume the user 110 did not collect any blue rocks, then in a regular gaming field, he would just be failing in completing the mission, and may receive suitable feedback about not collecting any blue rocks. However, according to the presently disclosed subject matter, the collection of the red rocks may later be analyzed with additional data, as detailed below, to determine low level of performance e.g., in lack of concentration skill or the user's lack of ability to discriminate between colors reliably.

Analysis module 224 can analyze the extracted at least one user measurement and the at least one activity measurement to obtain analyzed measurements (block 350). Analyzing together both the user measurements and the activity measurement can indicate on the user current participation in the game and his current status. One use of the resulting analyzed measurements is to adapt the experience while the user is participating based on his current participation. For example, is it determined from the user and activity measurements that the user fears, then a calming message can appear, or the virtual background can be changed. Adapting the experience is further described below.

The analyzed measurements can be measurements that are derived from either user measurements or the activity measurements or a combination of both. The user measurements and the activity measurements may be combined, aggregated, or manipulated in a different manner, in order to derive performance metrics, based on combination rules. User and activity measurements, e.g., taking the form of events, states, time series of values (continuous or categorical) can be analyzed in this step in various ways by applying typical operations for respective data types. Examples include:
applying temporal filters such as smoothing functions, calculating derivatives, averaging, low and high pass filtering etc.
transforming measurements by applying arithmetic operators or geometric transformations
calculating aggregations over time windows, e.g., counting number of specific events, averaging values
calculating combined measurements by applying arithmetic operators (e.g., calculating combined movement fluency measurement combining user measurement of body reflecting fluency of relevant body movements and activity measurement of fluency of movements of avatar moving through the game by calculating average of respective values)
calculating combined measurements by applying logical operators, e.g., create a trigger event if a certain game event (activity measurement: “close to virtual element in game”) and a certain movement is performed (user measurement: pinching gesture)
combining measurements by conditional filtering, e.g., only if game is in specific state as obtained from activity measurement (e.g., avatar near a wall) the motion of the arm (user measurement) is used to calculate the analyzed measurement to control the movement of the avatar.
applying thresholds to measurements to create trigger events (e.g., create trigger event “arm not fully stretched” if certain body angles as obtained by user measurements are below/above certain thresholds)
combinations of parts or all of the above processing steps, e.g., calculating a measurement reflecting overall activity level of the user grouped by different game states by applying various filters and transformation steps on user measurements of the human body and conditional filtering using activity measurements reflecting various game states. For example, user measurements of physical motions can be combined with activity measurements of non-stopping running of the game to determine a combined measurement of movement fluency.

Some examples of the analyzing measurements can include analyzing the following measurements extracted from the user measurements and the activity measurements:
An avatar 150 that falls while climbing the wall, the user 110 afterwards is frozen and maybe scared.
The avatar 150 arrives at a cliff and the user 110 is having an anxious facial expression.
While flying the user 110 is doing a lot of shaky/unstable movements.
Avatar 150 is hitting an object because of poor steering

In some examples, the resulting measurements may output a trigger or categorical/continuous values that drive the adaptation of the experience as explained further below. For example,
Triggering the experience: will cause something in the game to happen (make something appear/disappear, increase counter, . . . )
Categorical values: decisions with multiple option (different ways how the experience continuous, various ways to adapt the background, . . . )
Continuous value: Value that directly influences a game element (e.g. the speed of the character, the direction of the character).

In some examples, based on one or more of the analyzed user measurements, adapting module 226 may adapt at least one of the activities in the experience (block 352). In some examples, adapting the experience may be a result of the user 110 interacting with the virtual world included in the experience, and with virtual elements included in the experience. In order to determine the user 110 interaction with the virtual elements, and, optionally, to adapt the experience accordingly, real-time data may be obtained from the captured video, to define interactions with and/or control of the virtual elements in the experience, as displayed on the display 130. The data can be obtained e.g., by the image analysis described above and/or the analysis of the extracted human body as detailed above.

In some examples, the user 110 may control virtual elements in the experience. The experience can consequently be adapted. For example, at least one of the activities in the experience involves interaction with a plurality of virtual interactive elements. Adapting at least one of the activities may comprise changing the interactive elements in response to a user action, for example the experience may include an activity of collecting virtual rocks. Adapting the activity may comprise changing the interactive elements in response to a user action, for example, making the rocks disappear, once the user has collected them.

Another example of the user 110 controlling the virtual elements and adapting module 226 adapting the experience can be e.g., the human avatar 150 by human body motions, actions, or certain metrics. The avatar 150 can take various forms, including humanoid virtual avatars but also others, and may mimic one or more of the user's actions. The activity can be adapted by displaying the avatar 150 mimicking the user's action.

Other example of interaction with virtual elements can be virtual overlays that are shown as overlay on the image, which can be interacted with. This can also be interactions of a virtual element controlled by human motion with other virtual elements in a virtual world. Interactions with a virtual world can also be reactions of users to activities and events happening in the virtual world, for example, emotional elements in a story and measuring user reactions by means of body and facial analysis, and/or reaction times to certain triggers shown on the screen.

Adapting the activities based on the user interaction with the virtual world and elements may comprise changing a level of difficulty of at least one of the activities, replacing the virtual activities, activating and/or deactivating specific activities, adjusting at least one of the activities to assist the user in participating in the experience, providing guidance on the experience on the activity, adjusting or triggering audio output, or changing visual appearance of elements included in the experience, and other examples, or a combination of the above. For example, the analyzed measurements based on the activity measurements can indicate a potentially stressful situation in the game ahead for the user. The analyzed measurements based on the user measurements can indicate that the user feels stress. Hence, adaptation of the activity in this case can include replacing the current virtual background of an intense activity with a calmer background, or activating a calm voice voicing a calm message or guidance to the user.

Another example of adapting the experience would be replacing the current activity, including a certain task, with breathing exercises that the user 110 has to follow. Those versed in the art would appreciate that other examples for adapting an activity in an experience may be considered as part of the presently disclosed subject matter, such as providing feedback to the user to correct his physical motion.

In some cases, after the execution of a session of experience is completed, analysis module 224 can determine one or more combined measurements based on the analyzed measurements (block 360). In some examples, analyzing the measurements is performed concurrently as the user 110 participates in the game, while the experiences in the game are adapted. This facilitates that the experience that is executed responds to the user's current performance, and that the measurements that are analyzed include the user's performance in the adapted experience. In some examples, an additional, later, analysis is performed after the experience is completed. One purpose of the later analysis is to obtain parameters relating to human skills, and to derive performance metrics (that cover therapeutically relevant aspects (like movement fluency)), so, later, the level of performance of the skills and skill progression can be determined.

In some examples, the later analysis including determining the combined measurements with respect to the following parameters. Some of these parameters are also referred to during the analysis performed while the user participated in the experience:
Motion patterns: actions, correct/wrong, quality, speed, synchronicity, skills & abilities (balance, reach, . . . )
Reaction: cognitive (e.g., distraction), behavioral (e.g., inhibition), emotional (e.g., stress)
Performance related to abilities: cognitive (e.g., memory, planning, . . . ), physical
Aggregated: over multiple experiences (e.g., improvement, total exercise time)
Comparison with goals
analysis of reaction to a certain stimulus (physical, behavioural, emotional),
ability to control virtual elements through adequate and high quality body movements,
assessment of eye-hand coordination when controlling virtual elements through body,
assessment of planning abilities by analysis of execution of certain tasks.

In the above example of the user 110 climbing the mountain, the user measurements, including physical motions of the user of raising hands and legs, along with activity measurements of whether he reached the summit and how many rocks did the user 110 collect on his way up, can be analysed. For example, correct physical motions of climbing (user measurements), along with reaching the next level in the experience (activity measurement), may result in a combined measurement of success in reaching the next level when performing climbing.

On the other hand, correct physical motions of climbing (user measurements), along with failing to pass even the first stage of the climbing, may result in a different combined measurement of failure to meet the goals of the experience. Also, the event of encountering a dangerous obstacle in the experience can be combined with analysing the reaction of the user, e.g. does the user stop the movement and/or hesitate in order to think how to avoid it. Is there an anxious expression on the face? Another example, in the case of falling down while climbing the wall, does the user appear frustrated? (facial expression, body language)

Another example of a combined measurement is a lack of focus. This may have been derived from constantly moving the eye gaze to the side (a user measurement) when combined with a focused target the user 110 has to shoot at (an activity measurement). Yet another example can include a combined measurement of a positive emotion, when combining the physical emotion of rising the corners of the mouth with reaching an end of a level in the experience.

Below are some examples of how to determine on combined measurements:
Gating of a user measurement, based on an activity measurement: only aggregate data for a specific user measurement, if the current experience state requires the user to carry out the movement of interest. E.g., assume a performance level for the skill “arm reach” is desired. Among all user measurements, at this stage, only the measurements of the stretching of the arm is aggregated, when the game requires the user to do a climb-up movement that entails stretching of the arms.
Combining in-experience triggers with user measurements: assume there is a certain event in the experience, and the output to analysis is the reaction of the user to this event as measured by one or more user measurements, e.g., there is an event in the experience that may trigger fear, for instance arriving at a steep cliff. A level of a “fear index” can by calculated by combining the facial expression measurement (is there a fear full expression?) with the amount of movement of the users (is the user “freezing”?) within a short interval after the trigger event.
Combining in-experience objectives with user measurement: there is a certain objective in the experience, and the user must move in a certain way in order to reach the objective. A combined measurement can include a quality metric that calculates the optimality of the user measurement with respect to reaching the objective. For example, the in-game character needs to fly from A to B in the shortest possible path, and the flight path is controlled by the user measurements (e.g., by arm movements). A “motor control” skill can then be determined by calculating the difference between the optimal direction to reach B, and the actual flight direction as determined by the user input.
Correlation of activity and user measurements: one or more continuous activity measurements can be combined with one or more continuous user measurements to understand if they are statistically correlated. E.g. a speed measurement inside the experience can be correlated with the heart rate of the user. High correlation may indicate that the user has a low stress tolerance, as he is easily stressed by time pressure.

In some cases, based on one or more of the combined measurements, analysis module 224 can determine a level of performance of one or more human skills (block 370).

The level of performance can be any indication of the performance of the user in a skill or skills of interest. For example, the level of performance can be one or more scores derived from the combined measurements. The scores can be particular for each of the skills, or a fused score, e.g., a function of interim scores given to skills. The fused score may indicate a general level of performance of the user 110 in the experience, e.g. an assessment of the human skills. If the user 110 succeeded in the experience and presented high performance in several aspects during the participation, then then the fused score of his performance, e.g., the level of performance of the human, will be high.

In some examples, in order to determine the level of performance, the combined measurements can be aggregated to more high-level human skills, e.g., by mapping the combined measurements to one or more human skills of the user 110, and determining insights pertaining to human skills.

In some examples, a combination of one or more combined measurements can be mapped to one or more skills with a certain weighting function. In a non-limiting example, a simple weighted average function can be defined that maps combined measurements A, B, C to a skill S, e.g., as follows:

$$S = 0.2 * A + 0.3 * B + 0.5 * C$$

Another example of mapping the combined measurements into skills can be done by using non-linear combinations. For example, if combined measurement A<threshold, the skill S is always 0.

In some examples, rules mapping certain measurements to certain skills can be pre-defined and applied. For example, the skill “attention” can be seen as a combination from measurements “Composure/stillness” and “Sequencing””, with an equal weight.

The weighting function to determine level of performance of human skills can be pre-defined or learned/fine-tuned over time, e.g., with respect to each skill individually, or to a combination of skills, given the data of an individual user, the user 110, or a group of other users.

In some cases, skills can be arranged and defined hierarchically, e.g., balance, core strength, and upper limb strength, are aspects of gross motor skills, thus the level of performance for gross motor skills can be determined as a function of the level of performance of other focused skills.

In some examples, determining a level of performance of one or more human skills can include determining skill progression for one or more of the skills (block 372). Optionally, the analysis module 224 can determine the skill progression independently of determining the level of performance. As illustrated with respect to FIGS. 5 and 6, a skill 235a can be associated with one or more levels, where each level may be associated with one or more progress markers. Progress markers may be defined per skill level and may contain a set of criteria that need to be met for the progress marker to be considered to be achieved. These criteria can be rules on top of the underlying values considered in the progress marker (e.g., reach height>50% or error rate<20%) but also can contain criteria that consider values aggregated over certain time windows (e.g., average score for bi-lateral coordination>40% over last 10 games). To determine if a certain level was reached, the associated progress markers are evaluated by the system and a progress score for each progress marker is calculated. This progress score reflects to what extent the respective progress marker criteria are met. Progress scores of all relevant progress markers for a certain skill level may be aggregated to a progress score for the skill level (e.g., by taking the average of all progress scores for the individual progress markers). If the aggregated progress score for a certain skill level reaches 100%, the skill level is considered to be achieved. Determining the skill progression may be performed based on the combined measurement, on the activity measurement, on the user measurement or a combination thereof.

Below are some examples of user and activity measurements pertaining to interaction of the user 110 with the virtual world, which are analyzed, optionally, combined, that may then be used to determine the level of performance of certain human skills.
i. The reaction time to a suddenly appearing virtual object can be measured to understand the skill of cognitive processing speed.
ii. Analysis of the user 110 behavior around stressful events in the experience allows conclusions about stress tolerance.
iii. Analyzing movements close to adverse virtual objects in the experience can give insights on inhibition.

Following are some examples of experiences and relevant skills required to be improved by the user:

Following are three examples of experiences and measurements that can be extracted and analyzed. One example of an experience may include displaying a virtual environment where the user 110 has to climb a mountain. The set of skills of the user 110 that were defined to be improved may include: bi-lateral coordination, upper body strength, planning, and inhibition. The following data and measurements may be extracted from the captured video of the user 110 analyzed:
Quality (motor): analyzing if body movement was executed correctly, e.g., stable body posture, arms moving high enough, movement along body, etc.
Speed (motor): speed of expected body movement.
Movement fluency (motor): indicates if movement is executed in fluent way, or if there are interruptions.
Reach height (motor): symmetry of left and right arm reach.
Motion dissociation (motor): analyzing coordination between the two hands. One should stay still, while the other hand moves.

Analysis based on the above, and possibly other user and activity measurement, can be performed, a combined measurement can be determined, and a level of performance of the corresponding skills may then be determined:
Task completion (cognitive): analyzing to which extent the user completes the task within the game (collection of coins, avoiding obstacles/bombs, etc.).
Bi-lateral coordination (motor): analyzes coordinated reciprocal movement, e.g., left arm moves up when right arm moves down.
Planning (cognitive): analyzing climbing path compared to optimum, in order to assess planning skills.
Inhibition (behavioral): analyzing behavior when approaching dangerous objects along the way (e.g., lava), and whether the user slows down his movement to avoid hitting the obstacles.

Another example of an experience may include an environment where the user 110 has to control the path of an avatar on a surface by performing specific movements. The focus on the skills of the user that should be improved may include: balance, planning, and motor control.

The following data and measurements may be extracted from the captured video of the user 110, and may later be analyzed:
Fluency: indicates if movement is executed in a fluent way, or if there are interruptions, this may later indicate motor control skills.
Balance: measuring balance skills of user, analyzing his body posture and body movement patterns.
Motor control: analyzing motor control abilities of the user to navigate, and whether the user is able to steer in a direct and smooth manner towards the targets.

Analysis based on the above, and possibly other user and activity measurement, can be performed, a combined measurement can be determined, and a level of performance of the corresponding skills may then be determined:
Planning (cognitive): analysis of planning skills by comparing actual movement path of an astronaut avatar chosen compared to an ideal path.
Stress tolerance (emotional): analyzing reaction (facial, body, behavior) or other performance metrics in the game (fluency, balance, motor control, . . . ) if additional stress is induced by, e.g., showing oxygen tank is empty and user needs to collect next oxygen tank very quickly.
Spatial orientation (cognitive): analysis of spatial awareness within the game world (e.g., did the user get lost, go in the wrong direction, find a short path, . . . )
Frustration tolerance (emotional): analyzing the reaction (facial, body language, behavior) to failure or other frustrating situations.

Another example of an experience may include gliding, i.e., the user needs to control the flight path of the avatar by specific movements. The focus on the skills of the user that should be improved may include memory, sequencing, and movement isolation.

The following data and measurements may be extracted from the captured video of the user 110, and may later be analyzed:
Movement isolation: analyzing ability to only move desired body parts (e.g., stretched arm up and down) and not other parts (e.g., shoulder)
Symmetry: symmetry of left and right arm movement
Extroversion (emotional): game asks to “shake off the tension”, analyze the shyness/extroversion via body movement, facial expression.
Composure/stillness (motor/behavior): how much does a user move in front of the camera, when the game requires him/her to be still.
Reaction time/mental processing speed: measure time and correctness of an expected body action that should come after a trigger in the game.

Analysis based on the above, and possibly other user and activity measurement, can be performed, a combined measurement can be determined, and a level of performance of the corresponding skills may then be determined:
Behavior/personality traits: presents a challenging situation within the game story (you are bullied, you should console somebody, etc.), analyze the choice of reaction (body action, body language, facial).
Sequencing: analyzing memory and sequencing skills by analyzing user behavior to collect targets with regard to task (e.g., collected in right order, movement path)
Emotional intelligence: Allow control of certain game aspects via emotional facial expression (smile, frown, etc.).
Fine motor control: Analyze dexterity and action of hand movements, given a manipulation task within a game.
Focus/distraction (cognitive): Add distracting elements in the game and assess if other metrics are affected.
Prioritization (cognitive): Give simultaneous tasks that needs to be prioritized by the user, analyze task performance and reaction (body, facial).

In some examples, after the level of performance is determined, and optionally, also the skill progression, feedback module 225 can generating and provide feedback (block 380). For example, the feedback can include the level of performance, one or more skills, the current patient's level in the scale of levels, and optionally, its progress over the levels. The feedback can be transmitted to the user 110, relatives of the user 110, or a therapist related to the user 110, or any third party related to the user 110. Providing the feedback facilitates the user to improve the level of performance of the human skills by improving performance of at least some of the user measurements or by improving the user adapting the experience, e.g. by performing better with the avatar. The improvement later resulted in improvement in the combined measurements.

A feedback can be any insight that is generated based on the user measurements or the activity experience, or their combination, and may relate to participation of the user 110 in the experience. The feedback may pertain to any of the aspects of the measurement detailed above. In cases where at least one of the user actions comprises a body motion, then the generated feedback may comprise providing data relating to a more accurate version of the body motion. An avatar may be configured to illustrate the more accurate version of the body motion.

In some examples, based on expert knowledge (e.g., stored data of correct movements, stored metrics) or previously collected data, a feedback can include a certain recommendation of which skills should be worked on, which exercise should be next, or which certain configuration should be used for that exercise (e.g., difficulty level or certain restrictions). Based on the results of a plurality of users using assessment system 170, the recommendation and configuration might be adapted with respect to the running of the experience. Recommendations can also depend on status regarding skill levels, e.g., if certain levels were achieved or not as yet. Or if there was progress over a certain timeframe in a certain skill or not. Depending on determining the status or progress, there might be a recommendation to continue working on a specific skill or not.

In case an avatar is included in the experience, the feedback may comprise the avatar configured to illustrate the feedback. For example, if the feedback includes guidance on a more accurate version of the body motion, the avatar can illustrate the correct motion.

In some examples, metrics, analytics, and skills can be aggregated over multiple experiences to monitor progress of the user and to generate the feedback to the user 110, while considering that different experiences train different skills and expose different metrics, and that multiple metrics from multiple experiences may contribute to a specific skill with different relevance/weight. Also, the difficulty of the experience can affect how a metric should affect the skill aggregation. In some examples, the overall aggregation may ensure the computation of a consistent skill profile for the user 110, regardless of the experience/level of the experience.

Over time, the metrics and skills of a user 110 can be tracked to understand progress and how much the user has improved over time. For example, a skill profile can be maintained for a user 110 and may be repeatedly updated. Progress in one or more skill over time can be analyzed and visualized. Suitable feedback will then be generated and provided.

Optionally, the data which pertains to a user can be used together with other users' data to increase understanding and generate a more accurate feedback to the user.

In some examples, based on progress, the recommendation and configuration of the experience can be adapted repeatedly. The adaption may be derived from the level of performance, particular progress in skill levels, levels' progress markers or the feedback that was generated. Adaptation can be performed in a similar manner to that described above during the user participation in the experience. Knowledge of age or the user or condition (in case if user is a patient) may affect the presentation and calculation of the data.

In some examples, processor 220 can repeatedly execute the stages while the game is running, e.g., in real time, where the user's movements and exercises are repeatedly captured by the camera 140 when participating in the game, the captured video is analyzed, combined measurements are determined, and feedback is provided, while the user continues to participate in the game. Feedback module 225 can provide immediate feedback and guidance to the user 110 or to another third party related to the user 110, in real-time, during a certain exercise that the user 110 performed, e.g., if a motion is not performed correctly, visual or auditory feedback can be given to the user. Optionally, the processor 220 can repeatedly execute the stages 310-380, while providing feedback to the user 110, optionally, adapt the experience or the treatment plan based on the level of performance and optionally the skill progression and/or the feedback, as later will be explained and then proceed to provide the same or adapted experience to the user 110, so over time the progress of the user in performing human skills can be tracked.

In cases where one wishes to assist the user 110 to enhance a specific set of one or more human skills, a personalized treatment plan may be generated for the user constituting a patient. An experience with at least some of the activities corresponding to the specific set of one or more human skills may be selected and provided to the user 110. The personalized treatment plan can be generated to facilitate the user to improve a set of human skills by improving the performance of at least some of the user measurements. The performance of each skill can be analysed based on the skill's levels, and the associated progress markers of each level.

The treatment plan may be fully defined by a therapist treating the user 110, or may be automatically generated, e.g., based on data pertaining to the user, for example, based on a set of one or more skills for improvement associated with the user 110, personal data such as gender, age, and history of experiences. Alternatively, or additionally, the user 110 may participate in one or more experiences, and, based on his participation in the experiences and the extracted measurements, a treatment plan may be automatically generated. The generated treatment plan can be personalized, such that it addresses a specific set of human skills that were identified as requiring improvement by the user 110. A treatment plan can be personalized, based on one of, or a combination of, at least the following: skills that were identified that were required to be improved, other user data known diagnosis or conditions, or measurements taken by the system, and other factors.

Yet alternatively or additionally, a sequence of recommendations generated based on the feedback, as detailed above, can be concatenated to form an automatically generated treatment plan personalized for the user 110. Alternatively, or additionally, a treatment plan can also be implicit, e.g., rather a flow through the different experiences that is adapted, based on intermediate results.

Based on the user's performance and the skill progression, the treatment plan, and, accordingly, the experience and activities in the experiences, can be updated and adapted to the user's level and progress, resulting in enhancing the overall improvement of the skills of the user. The experiences may be adapted to address the determined improvement of certain human skills of the user.

As mentioned above, the assessment system 170 can communicate with third parties related to the user 110, such as a therapist. In some examples, the therapist may be involved in personalizing treatment plans, including that which was generated for the user 110, and can influence the recommended configuration to an arbitrary extent, as the therapist sees fit. As described, a therapist can determine a treatment plan for the user 110, by determining a set of skills from skills 235a-235n that were identified as requiring improvement. The therapist may also define a certain level of a certain skill as a goal for the user 110. After receiving the feedback, the therapist can also recommend on configuration for experiences in the game. Hence, the therapist may send a modification input, e.g., to adapting module 226, to adapt the experience accordingly. Adapting module 226 can adapt at least one activity and/or the experience in accordance with the modification input of the therapist in a manner similar to that described above with respect to adapting the experience during participation in the experience. Adapting the at least one activity and/or experience may include executing any of the actions detailed above with respect to block 332 in FIG. 3.

In addition to therapist involvement in the treatment plan, and adapting the experience based on the feedback the therapist received, interactions of the therapist with the assessment system 170 can be tracked and used as expert knowledge, e.g., by learning, based on which skill profile a therapist recommended, and which follow-up treatment. This data can be used to improve recommendations made by the system 170. Optionally, meta-analysis of the value of the therapist recommendations can be monitored, e.g., by checking to which extent recommendations of the therapist helped to improve the metrics and skills of a user 110. Direct and immediate analysis of the therapist, e.g. during the user is participating the experience is likewise optional

Executing a personalized treatment plan is advantageous and facilitates motivating real-time experiences of the patient in the treatment plan, while tracking and monitoring the execution in a privacy-aware manner. Motivation of the user to perform the exercises may be increased, as the required exercises are tied to a game, an entertainment experience, the avatar's actions are affected by the user's movements, and the user may feel an immersive experience in a virtual environment, where the user has a representation of himself in the game.

The feedback, as well as various metrics, may assist in objectively measuring the progress of the patient, to get a detailed status of the treatment plan and the treatment. In addition, the system 170 may communicate, optionally, in real-time, with a therapist who can view the user's motions, correct, and adjust the treatment plan, or provide additional feedback to the user 110. In such a manner, the method facilitates dynamic adaptation of the treatment plan, resulting in faster progress of the patient in the treatment plan.

It should be noted that recitation of a treatment plan should not be considered as limiting. A treatment plan can also be implicit. Those skilled in the art will readily appreciate that the teachings of the presently disclosed subject matter are, likewise, applicable to a flow through different experiences that is adapted based on intermediate results. For example, the exercises can be adapted automatically and dynamically, e.g., by reducing/increasing difficulty levels, changing the recommendation based on performance, etc.

The method described above may be advantageous and may be used to enable an interactive experience of the patient on multiple levels:
I. Interactive control, e.g., control the experience with any measurements described above;
II. Dynamic game experience: Change experience (visuals, difficulty, . . . ) dynamically based on any of the measurements described above;
III. Live feedback: direct feedback is provided while participating in the game, (e.g., “try to keep your upper body straight”). Some more complex feedbacks may be provided e.g., quality of motion was good to create positive reinforcement for learning/improvement;
IV. Using the assessment in order to dynamically adapt the therapy plan and sequence, e.g., by suggesting different follow-up games and levels.

It should be noted that the method does not necessarily require a predefined treatment plan to be associated with a patient, and those versed in the art would realize that patients can also do exercises to improve their skills without a treatment plan. The assessment done by the system will help to automatically recommend best experiences to improve further, address a specific issue/skill, and to manage progress in a personalized manner tailored to the individual.

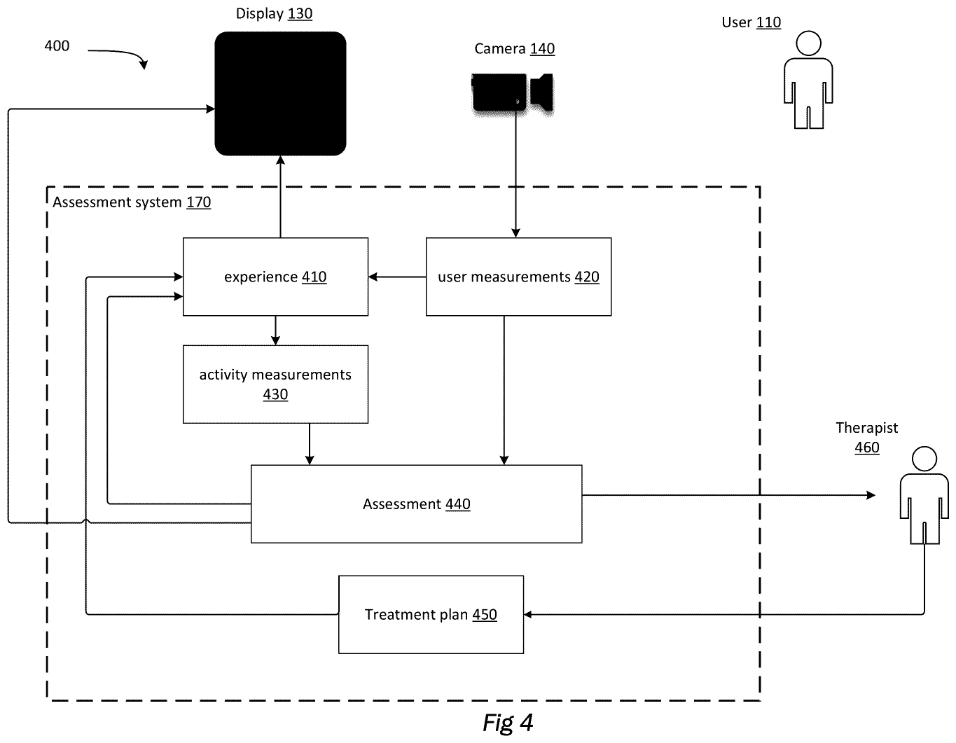

FIG. 4 illustrates a generalized flow of data between entities and components in assessment environment 100 in FIG. 1, according to certain embodiments of the presently disclosed subject matter.

As illustrated, the user 110 participates in a game experience 410, as displayed on a screen, illustrated by display 130. The user 110 may be captured by the camera 140. In some examples, the screen and camera can be embedded in a single user device 120 as illustrated in FIG. 1. The human body of the user 110, as captured by a video by the camera, may be analyzed in user measurements 420 to extract user measurements, e.g., body motions, facial muscles & expressions, and internal functions.

The experience, including a plurality of activities, may also be analyzed e.g., in experience measurements 430, by analyzing events that occurred in the game, states, triggers, collected items, events including timestamps, etc. User measurements 420 as well as activities measurements 430 may be implemented by obtaining module 222 and extraction module 223 described above with reference to FIG. 2.

Both the user measurements 420 and the activity measurements 430 may be analyzed together, e.g., in assessment 440 implemented by analysis module 224 of FIG. 2. As explained above, the analysis may include analysis of motions, reaction of the user to the activities in the experience, and other relevant measurements and their combinations.

The results and any feedback generated by the assessment 440 may be transmitted to a therapist 460, who may adapt and update any treatment plan that may exist for the user 110 and/or adapt experience or activities and goals to achieve in the experience.

The method and data flow described above and in relation to FIG. 2 and FIG. 3 may be performed by elements illustrated in a functional block diagram of assessment system 170, as illustrated in FIGS. 1 and 2, in accordance with some embodiments of the present invention, for example, by processing and memory circuitry (PMC) 420 comprising obtaining module 422 which is configured to obtain the captured video of the user, analyzing module 424 configured to analyze the captured video, and feedback module 426 configured to provide feedback to the user.

It is noted that the teachings of the presently disclosed subject matter are not bound by the flow chart or data flow illustrated in FIGS. 3 and 4, and that the illustrated operations can occur out of the illustrated order. For example, operations <330> and <340> or <332> and <340> shown in succession can be executed substantially concurrently, or in the reverse order.

It is to be understood that the invention is not limited in its application to the details set forth in the description contained herein or illustrated in the drawings. The invention is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the presently disclosed subject matter.

It will also be understood that the system according to the invention may be, at least partly, implemented on a suitably programmed computer. Likewise, the invention contemplates a computer program being readable by a computer for executing the method of the invention. The invention further contemplates a non-transitory computer-readable memory tangibly embodying a program of instructions executable by the computer for executing the method of the invention.

Those skilled in the art will readily appreciate that various modifications and changes can be applied to the embodiments of the invention as hereinbefore described without departing from its scope, defined in and by the appended claims.

1. A computer-implemented method for determining performance of human skills of a user, the method comprising:
providing on a user device an interactive virtual experience in which the user can participate, the experience comprising one or more activities;
while the user participates in the experience, repeatedly executing:
obtaining camera output comprising a video of the user performing one or more user actions;
extracting one or more user measurements from the one or more user actions;
extracting one or more activity measurements from the provided experience;
analyzing at least one of the one or more user measurements and the one or more activity measurements to obtain analyzed measurements; and
adapting at least one of the activities in the experience based on at least the analyzed measurements; and
determining one or more combined measurements based on the analyzed measurements; and
determining level of performance of one or more human skills based on the one or more combined measurements.

2. The method of claim 1, the method further comprising:
based on the level of performance, generating feedback; and
providing the feedback;
whereby the provided feedback facilitates the user to improve the level of performance of the human skills by improving performance of at least some of the user measurements.

3. The method of claim 1, wherein at least one activity measurement is not related to success of the user in completing goals of the experience but is used in the determining the level of performance.

4. The method of claim 1, wherein adapting at least one of the activities comprises performing at least one of the following: changing a level of difficulty of at least one of the activities, replacing virtual activities included in the experience, activating and/or deactivating specific activities, adjusting at least one of the activities to assist the user in participating in the experience, providing guidance on experience in the activity, adjusting or triggering audio output, or changing visual appearance of elements included in the experience.

5. The method of claim 1, wherein at least one of the activities involves interaction with a plurality of virtual interactive elements, and wherein adapting at least one of the activities comprises changing the interactive elements in response to a user action.

6. The method of claim 5, wherein at least one element includes an avatar configured for mimicking at least one of the user actions.

7. The method of claim 1, wherein the one or more user measurements comprise a body motion.

8. The method of claim 1, wherein the one or more user measurements comprise facial muscles and expressions.

9. The method of claim 1, wherein the one or more user measurements comprise internal functions.

10. The method of claim 1, wherein the one or more activity measurements relate to continuous running of the experience.

11. The method of claim 1, wherein analyzing the one or more user measurements comprise comparing at least one of the extracted user measurements to a corresponding reference user measurement.

12. The method of claim 1, wherein extracting the one or more activity measurements comprise measuring one or more of performance, goals, or events within the experience, or a combination thereof.

13. The method of claim 1 wherein the analyzing comprises analyzing the one or more user measurements and the one or more activity measurements.

14. The method of claim 2, further comprising:
providing the feedback to a third party related to the user.

15. The method of claim 2, wherein at least one of the user actions comprises a body motion, and wherein the feedback comprises data relating to a more accurate version of the body motion.

16. The method of claim 15, wherein the feedback comprises an avatar configured to illustrate a more accurate version of the body motion.

17. The method of claim 2, wherein the experience is selected based on a personalized treatment plan generated to facilitate the user to improve one or more human skills by improving performance of at least some of the user measurements, wherein at least some of the activities in the selected experience correspond to the one or more human skills.

18. The method of claim 17, wherein the treatment plan is automatically generated, based on data pertaining to the user.

19. The method of claim 17, the method further comprising:
providing the feedback to a third party related to the user, wherein the third party is a therapist involved in generating treatment plans;
in response to providing the feedback, receiving a modification input from the therapist; and
adapting at least one of the activities and/or the experience in accordance with the modification input.

20. The method of claim 19, wherein the modification input includes data pertaining to modification in the personalized treatment plan, and prior to the modifying, the method further comprising:
based on the modification input, updating the treatment plan; and
adapting at least one of the activities and/or the experience, based on the updated treatment plan.

21. The method of claim 20, wherein adapting at least one of the activities and/or the experience comprises performing at least one of the following: changing a level of difficulty of at least one of the activities, replacing the virtual activities, activating and/or deactivating specific activities, adjusting at least one of the activities to assist the user in participating the experience, providing guidance on the experience on the activity, adjusting or triggering audio output, or changing visual appearance of elements included in the experience.

22. The method of claim 1, wherein the user device is a mobile user device comprising a camera.

23. The method of claim 1, wherein determining the one or more combined measurements comprises processing the analyzed user measurements and the activity measurements in order to assess one or more of the following aspects pertaining to human skills: motion patterns, reaction, performance related to abilities, aggregated measurements over a plurality of experiences, comparison with goals, cognitive and physical abilities, social skills, and behavioral skills.

24. The method of claim 1, wherein the analyzing comprises analyzing one or more user measurements using at least one of image analysis and analysis of extracted human body.

25. The method of claim 1, wherein determining the level of performance comprises mapping the combined measurements to the one or more human skills.

26. The method of claim 1, further comprising:
determining skill progression for one or more of the human skills.

27. The method of claim 26, wherein determining the skill progression for at least one of the human skills comprises determining the progress in one or more progress markers associated with levels of the at least one skill.

28. The method of claim 27, wherein the progress levels are selected from a group comprising: completion rate and level success, skill level, effort, counts, metrics or a combination thereof.

29. The method of claim 27, wherein a level is considered as reached, after all progress markers associated with the level are met.

30. A computerized system for determining performance of human skills of a user, the system comprising a processing and memory circuitry (PMC) configured to:
provide, on a user device, an interactive virtual experience in which the user can participate, the experience comprising one or more activities;
while the user participates in the experience, the PMC is configured to repeatedly execute:
obtain camera output comprising a video of the user performing one or more user actions;
extract one or more user measurements from the one or more user actions;
extract one or more activity measurements from the provided experience;
analyze at least one of the one or more user measurements and the one or more activity measurements to obtain analyzed measurements; and
adapt at least one of the activities in the experience based on at least the analyzed measurements; determine one or more combined measurements based on the analyzed measurements; and
determine level of performance of one or more human skills based on the one or more combined measurements.

31. A non-transitory computer readable storage medium tangibly embodying a program of instructions that, when executed by a computer, cause the computer to perform a method for determining performance of human skills of a user, the method comprising:
providing, on a user device, an interactive virtual experience in which the user can participate, the experience comprising one or more activities;
while the user participates in the experience, repeatedly executing:
obtaining camera output comprising a video of the user performing one or more user actions;
extracting one or more user measurements from the one or more user actions;
extracting one or more activity measurements from the provided experience;
analyzing the one or more user measurements and the one or more activity measurements to obtain analyzed measurements; and
adapting at least one of the activities in the experience based on at least the analyzed measurements;
determining one or more combined measurements based on the analyzed measurements; and
determining level of performance of one or more human skills based on the one or more combined measurements.

32. In a user device, a computer-implemented method for determining performance of human skills of a user, the method comprising:
providing an interactive virtual experience in which the user can participate, the experience comprising one or more activities;
displaying the experience;
while the user participates in the experience, repeatedly executing:
capturing, by a camera included in the user device, camera output of the user performing one or more user actions;
extracting one or more user measurements from the one or more user actions;
extracting one or more activity measurements from the provided experience;
analyzing the one or more user measurements and the one or more activity measurements obtain analyzed measurements;
adapting at least one of the activities in the experience based on at least the analyzed measurements;
determining one or more combined measurements based on the analyzed measurements; and
determining level of performance of one or more human skills based on the one or more combined measurements.

33. The method of claim 32, wherein providing data comprises:
based on one or more parameters relating to human skills, generating feedback; and
providing the feedback;
whereby the provided feedback facilitates the user to improve the level of performance of the human skills by improving performance of parameters indicative of human skills.
